(19) United States
(12) Patent Application Publication
(10) Pub. No.: US 2022/0203138 A1
(43) Pub. Date: Jun. 30, 2022

(54) Systems and Methods for Cancer Treatment

(71) Applicant: Mechanobiologics LLC, Galveston, TX (US)

(72) Inventors: Ajay Sanjay Tijore, Galveston, TX (US); Felix Margadant, Galveston, TX (US); Mingxi Yao, Galveston, TX (US); Michael P. Sheetz, Galveston, TX (US)

(21) Appl. No.: 17/607,819

(22) PCT Filed: Apr. 28, 2020
(86) PCT No.: PCT/US2020/030288

§ 371 Date: Oct. 29, 2021

Related U.S. Application Data
(60) Provisional application No. 62/841,520, filed on May 1, 2019.

(51) Int. Cl. A61N 7/00

(52) U.S. Cl. CPC A61N 7/00; A61N 2007/0004

(57) ABSTRACT

Methods and systems are provided for treating target cells in a target tissue. The method comprises: determining frequency and magnitude of a periodic force to be applied to the target cells to trigger a mechanically-induced apoptotic process in the target cells; and generating a sequence of programmed cycles of waves to the target tissue to apply the periodic forces to the target cells for a period of time. In some cases, the frequency, magnitude and the period of time are determined based at least in part on the type of target cells or mechanical properties of the target cells.

### CROSS-REFERENCE

This application claims priority to U.S. Provisional Patent Application No. 62/841,520, filed May 1, 2019 which is entirely incorporated herein by reference.

### BACKGROUND

Noninvasive cancer therapy requires high specificity where the treatment selectively targets cancer cells over normal tissues. In particular, it is desired to selectively kill malignant cancer cells over normal somatic cells. In the past, methods were developed to halt or destroy the tumor by selectively killing cancer cells. For instance, it is known that cell death can be regulated through distinct, and sometimes overlapping, signaling pathways in non-transformed cells. Manipulation of signaling pathways involved in cell death is an effective approach in treatment cancer. In most of those cases, treatments may involve using chemotherapeutic drugs to disrupt signaling pathways to eventually induce cell death. However, this chemo-treatment is often accompanied by serious side effects. In another example, application of high frequency ultrasound can provide high temperatures to the diseased tissue for ablation and/or tumor cell death due to high temperatures. However, high temperatures may also be generated in surrounding tissues such as normal tissue, skull, skin, which poses a problem for the patient being treated by creating unnecessary medical risks. It is desired to have an effective method to induce cancer cell death with improved selectivity as well as reduced side effects.

### SUMMARY

The present disclosure provides novel methods, devices and systems for inducing cell death by regulating mechanical-sensing signals, which can provide a less cytotoxic but effective way for treating diseases such as cancer. Methods and systems of the present disclosure may provide high-selectivity cancer treatment without introducing side effect. The methods and systems provided herein may address various drawbacks of conventional systems, including those recognized above. In some cases, these systems and methods may be capable of performing the novel treatment to trigger the apoptosis induced-cancer cell death in an automated and controlled manner. The provided in situ cancer treatment can be well suited for various kinds of cancer. The treatment involves generating and imparting periodic forces to diseased tissue to introduce apoptosis only in cancer cells while promoting growth in normal cells. This may beneficially improve the efficiency and efficacy of cancer treatment with drastically reduced side effect.

The process of recognizing and responding to mechanical stimuli is critical for growth and function of cells. Cells can respond to mechanical signals in the form of forces applied externally or generated by cell-matrix and cell-cell contacts. Some of those mechanical signals can further regulate the cell death process. Cancer cells and normal cells respond to mechanical stimuli differently. Cancer is a disease where the cells exhibit altered biomechanical properties. Early studies determined that cancer cells grew on soft agar whereas normal cells did not. More recent studies show that more than seventy-five percent of 40 randomly selected cancer lines lack the ability to sense rigidity of surrounding matrices.

It is observed that apoptosis induced-cancer cell death can be triggered by periodic stretching of the cancer cells. Unlike conventional ultrasound ablation which causes cell death due to heat or high temperature, cancer cells treated by the provided methods and systems apoptose after low intensity, long periods of mechanical stimuli thereby reducing the damage to normal cells due to either thermal effects and/or cavitational effects. In particular, periodic forces are generated in a combination of duration, magnitude, and frequency that are sufficient to distort the internal cell organelles such that a mechanically-induced apoptotic process is triggered, while the generated periodic forces are small enough such that the normal cells and healthy tissues are not subjected to mechanical damage.

In one aspect of the present disclosure, the present disclosure provides a method for treating target cells in a target tissue. The method comprises: determining frequency and magnitude of a periodic force to be applied to the target cells to trigger mechanically-induced apoptotic process in the target cells; and generating a sequence of programmed cycles of waves to the target tissue to apply the periodic forces to the target cells for a period of time. In some case, the frequency, magnitude and the period of time are determined based at least in part on a type of the target cells or mechanical properties of the target cell.

In an aspect of the present disclosure, a method is provided for treating target cells in a target tissue. The method comprises: determining frequency and magnitude of a periodic force to be applied to the target cells to trigger a mechanically-induced apoptotic process in the target cells; and generating a sequence of programmed cycles of waves to the target tissue to apply the periodic forces to the target cells for a period of time, where the frequency, magnitude and the period of time of the periodic force are determined based at least in part on a type of the target cells or mechanical properties of the target cells.

In some embodiments of the method, the target cells are cancer cells and wherein the periodic force is imparted on both the cancer cells and normal cells surrounding the cancer cells. In some cases, the periodic force promotes or preserves survival and regeneration of the normal cells.

In some embodiments, the frequency of the periodic force is in a range of 30 kHz to 250 kHz. Alternatively, the frequency of the periodic force is in a range of 5 kHz to 50 kHz or in a range of 150 kHz to 1 MHz.

In some embodiments, the target cells are deformed periodically at the frequency for the period of time such that the mechanically-induced apoptotic process is triggered. In some embodiments, the sequence of programmed cycles of waves are ultrasound waves and are generated by an ultrasound generator. In some embodiments, the sequence of programmed cycles of waves are generated by at least one of ultrasound generator, ultrasound transducer, microwave amplification by stimulated emission of radiation (MASER) generator, magnetic resonance imaging (MRI) device, positron emission tomography (PET) device, near infrared light source, large area generator, and neutral generator.

In some embodiments, the sequence of programmed cycles of waves are mechanical waves, electromagnetic waves, periodic pneumatic or hydraulic pressure waves. In some embodiments, the sequence of programmed cycles of waves are a composite waveform including multiple frequency components. In some embodiments, at least one of the multiple frequency components has a frequency higher than the frequency of the periodic force. In some embodiments, the mechanical properties of the target cells or target tissue comprise a resonant frequency of the target cells or an inertia of the target cells.

In a related yet separate aspect, a system is provided for treating target cells in a target tissue. The system comprises: (i) an energy source configured to generate a sequence of programmed cycles of waves to the target tissue to apply a periodic force to the target cells for a period of time; and (ii) one or more processors programmed to determine the frequency and magnitude of the periodic force applied to the target cells to trigger mechanically-induced apoptotic process in the target cells, wherein the frequency, magnitude and the period of time are determined based at least in part on a type of the target cells or mechanical properties of the target cell.

In some embodiments, the target cells are cancer cells and wherein the periodic force is imparted on both the cancer cells and normal cells surrounding the cancer cells. In some cases, the periodic force promotes or preserves survival and regeneration of the normal cells.

In some embodiments, the frequency of the periodic force is in a range of 30 kHz to 250 kHz. Alternatively, the frequency of the periodic force is in a range of 5 kHz to 50 kHz or in a range of 150 kHz to 1 MHz.

In some embodiments, the target cells are deformed periodically at the frequency for the period of time such that the mechanically-induced apoptotic process is triggered. In some embodiments, the sequence of programmed cycles of waves are ultrasound waves and are generated by an ultrasound generator. In some embodiments, the sequence of programmed cycles of waves are generated by at least one of ultrasound generator, ultrasound transducer, microwave amplification by stimulated emission of radiation (MASER) generator, magnetic resonance imaging (MRI) device, positron emission tomography (PET) device, near infrared light source, large area generator, and neutral generator.

In some embodiments, the sequence of programmed cycles of waves are mechanical waves, electromagnetic waves, periodic pneumatic or hydraulic pressure waves. In some embodiments, the sequence of programmed cycles of waves are a composite waveform including multiple frequency components. In some embodiments, at least one of the multiple frequency components has a frequency higher than the frequency of the periodic force. In some embodiments, the mechanical properties of the target cells or target tissue comprise a resonant frequency of the target cells or an inertia of the target cells.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein) of which:

FIG. 1 schematically shows an example of cyclic forces experienced at the target tissue or target cells;

FIGS. 2A-2C schematically shows a device for performing cancer treatment by implementing a method provided herein;

FIG. 3 schematically shows a system in which cancer treatment method described herein may be implemented.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein and in the appended claims, the singular forms “a,” “and,” and “the” include plural referents unless the context clearly dictates otherwise. Thus, for example, reference to “a programmed cycle” includes a plurality of programmed cycles, reference to “a wave” includes a plurality of waves, and reference to “the signaling pathway” includes reference to one or more signaling pathways (or to a plurality of signaling pathways) and equivalents thereof known to those skilled in the art, and so forth.

The term “about” when referring to a number or a numerical range means that the number or numerical range referred to is an approximation within experimental variability (or within statistical experimental error), and thus the number or numerical range may vary between 1% and 15% of the stated number or numerical range.

When a range of values is provided for describing properties, such as frequencies, or penetration distances/depth, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure provided herein.

The term “and/or” as used herein is a functional word to indicate that two words or expressions are to be taken together or individually. For example, A and/or B encompasses A alone, B alone, and A and B together.

The term “comprising” (and related terms such as “comprise” or “comprises” or “having” or “including”) is not intended to exclude that in other embodiments, for example, an embodiment of any composition of matter, composition, method, or process, or the like, described herein, may “consist of” or “consist essentially of” the described features.

The term “treating” refers to inhibiting, preventing, curing, reversing, attenuating, alleviating, minimizing, suppressing or halting the deleterious effects of a disease and/or causing the reduction, remission, or regression of a disease. Those of skill in the art will understand that various methodologies and assays can be used to assess the development of a disease, and similarly, various methodologies and assays may be used to assess the reduction, remission or regression of the disease.

### Method

Cell death is a critical and active process which maintains tissue homeostasis and eliminates potentially harmful cells. There are three major types of cell death including apoptosis, autophagic cell death and necrosis. The provided systems and methods may beneficially treat tumor or cancer by introducing cancer cell death in a controlled manner. In particular, the treatment systems or methods may be non-invasive and capable of targeting cancer cells with improved selectivity and little damage to normal cells. For example, the treatment systems or methods may involve using an ultrasonic device to effect apoptosis-induced cell death, while preserving the survival and growth of normal cells in a controlled manner. The efficacy of the treatment is highly selective between the diseased cells and normal cells that only cancerous cells are killed while normal cells are unharmed and may even grow.

In one aspect of the present disclosure, a method is provided for generating and imparting periodic forces to cancer cells in situ thereby treating cancer. The method may comprise applying one or more programmed cycles of waves to the target cell for a pre-determined period of time. The programmed cycles of waves impart periodic forces with controlled duration, magnitude, and frequency that are sufficient to distort the cancer cells and internal organelles such that a mechanically-induced apoptotic process is triggered. The generated periodic forces may have low intensity and be brief enough such that the normal cells and healthy tissues surrounding the cancer cells being treated may not be subjected to mechanical or thermal damage.

In some cases, the frequency and magnitude of the periodic forces may be pre-determined based on the types of cells or target disease. In some cases, the programmed cycles of waves may be applied to the target cells at a frequency in a range of about 30-250 kHz, with low intensity for a long duration of time (e.g., hours) so as to introduce apoptosis by calcium mediated-calpain activation in the target (e.g., cancer) cells meanwhile not damaging normal cells. The programmed cycles of waves can be in any range below 30 kHz or above 250 kHz so long as the periodic forces imparted on the target tissue (e.g., internal organelles) mechanically distort the cancer/normal cells such that a mechanically-induced apoptotic process is triggered in the cancer cells.

The term “spontaneous cell death” as used herein includes but is not limited to apoptosis, autophagy, and certain forms of necrosis. In the present disclosure, the spontaneous cell death may be caused by a periodic and repetitive sequence of forces which is different from cell death caused by increased amplitude or intensity as in the convention ablation treatment.

The term “apoptosis” as used herein may refer to a regulated network of biochemical events which eventually leads to cell suicide, and is characterized by readily observable morphological and biochemical phenomena, such as fragmentation of the deoxyribonucleic acid (DNA), condensation of the chromatin, chromosome migration in cell nuclei, the formation of apoptotic bodies, mitochondrial swelling, and the like. Inhibition or dysregulation in apoptotic cell death machinery is a hallmark of cancer cells, which is responsible for both tumor development and progression. The provided methods and systems are capable of re-activating the lost apoptotic mechanisms in the transformed cells.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny from a biological entity obtained in vivo or cultured in vitro are also encompassed.

The target cells used in the method of the present disclosure can be any cells that are dysregulated in cell death process. In some embodiments, the target cells are a cancer cells or transformed cells. Cancer cells that can be used in the method of the present disclosure includes, but is not limited to, prostate cancer cells, breast cancer cells, colon cancer cells, lung cancer cells, head & neck cancer cells, brain cancer cells, bladder cancer cells, lymphocytes, ovarian cancer cells, renal & testis cancer cells, melanoma cancer cells, liver cancer cells, cervical cancer cells, pancreatic cancer cells or gastrointestinal cancer cells.

In some embodiments, the method of the present invention does not induce a spontaneous cell death in normal cells. The term “normal cell” as used herein refers to the basic healthy cell or non-transformed cell with normal functions to maintain correct functioning of tissues, organs, and organ systems. Normal cells will undergo spontaneous cell death as part of normal development, to maintain tissue homeostasis, and in response to unrepairable damage.

Target tissues or target regions under the provided treatment may comprise both the target cells, i.e., cancer cells, and normal cells. The present treatment method may be effective at the cell-level such that when the cyclic waves or cyclic forces are applied to the target tissue, only the target cells are killed due to mechanically-induced apoptotic processes whereas cell viability is enhanced/preserved for normal cells.

In some embodiments, the method of the present disclosure may induce or regulate apoptosis of the target cell by exposing the target cell to periodic stretch/pressure forces with pre-determined characteristics. The methods and systems may be based on the observation that mechanically-induced stress activates calcium channels to overload cancer cells with Ca′ ions and triggers cell death via apoptosis. In some embodiments, the method of the present disclosure at least partially stimulates, increases, opens, activates, facilitates, enhances activation, sensitizes, or upregulates apoptotic signaling pathways of the target cells. In some embodiments, the method of the present disclosure activates the cell surface receptors involved in apoptosis signaling pathways of the target cell. The cell surface receptors may be capable of sensing mechanical cues, radiation or shock waves. The provided methods and systems may apply cyclic and/or structured remote force to the target tissues which effectively alters cancerous cells activity and leads the cancerous cells into apoptotic processes.

The term “autophagy” as used herein refers to a process by which cytoplasmic material is delivered to lysosomes for degradation. During this process, an autophagosome having a double membrane encloses the components of the cell to be degraded, and the autophagosome then fuses with a lysosome which carries out the function of degradation and results in the recycling of cellular materials. Autophagy-lysosome pathway and the ubiquitin-proteasome pathway are two principal pathways in autophagy processes and these processes are also closely associated with apoptosis. Autophagy appears to have great significance for the treatment of various diseases caused by misfolded protein aggregates in specific tissues and cells such as cancers.

In some embodiments, the method of the present disclosure induces or regulates autophagy of the target cell. In some embodiments, the method of the present disclosure at least partially stimulates, increases, opens, activates, facilitates, enhances activation, sensitizes, or upregulates the autophagy signaling pathway of the target cell. In some embodiments, the method of the present disclosure activates the cell surface receptors involved in the autophagy signaling pathway of the target cell. In some embodiments, the cell surface receptors are mechanical sensing receptors. In some embodiments, the cell surface receptors are radiation sensing receptors. In some embodiments, the cell receptors are shock sensing receptors.

The mechanically-induced stress to activate calcium channels may be applied in the form of cyclic waves or cyclic forces. The cyclic forces may be delivered to the target tissue in the form of ultrasound waves or pulses. The “wave” as used herein refers to any wave with pre-determined characteristics such as frequencies, energy, intensity, or duty factor that upon application to a target tissue or target region may deform the cells periodically thereby inducing spontaneous cell death of a target cell. The aforementioned characteristics may be determined such that force may be transduced to the target region to induce periodic membrane stress rather than molecular heating or a static deformation thereby avoiding physical damage to normal cells. In some cases, the force wave or pulses may be administered to the target region by penetrating the skin, bone, muscle, and underlying fascia without inducing damage to any portion of human body other than killing the target cancerous cells within the target region. In some cases, the force wave may be transmitted in the form of unfocussed ultrasound waves. Alternatively or in addition to, the force wave may be transmitted in the form of focused ultrasound wave to focus on the target region/tissue with reduced impact to the untargeted region/tissue.

The various characteristics of the cyclic force such as intensities, frequencies, amplitude and the like may refer to the intensity, frequency or amplitude levels at the effective tissue site. As an example, the force may comprise low intensity cycles applied to the tissue site over hours. In some cases, the cyclic force effected directly on the tissue site or target cells may be delivered with long irradiation times (e.g., hours) at low-frequency (e.g., 5-30 kHz, 30-250 kHz, 150 kHz to 1 MHz, etc.) and at low intensity levels (e.g., <500 mW/cm²). The cyclic forces may also be referred to as wave, waveform, structured force, structured waveform and the like which are used interchangeably throughout the specification unless context suggests otherwise.

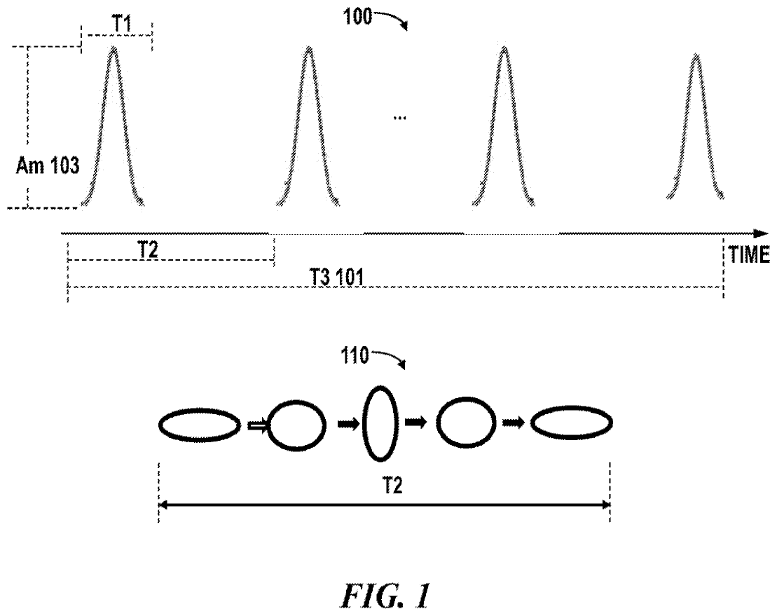

FIG. 1 schematically shows an example of cyclic forces (100) experienced at the target tissue or target cells. In some cases, the one or more cycles of the waves applied to a target region may have constant frequency. In some cases, the one or more cycles of the waves may have variable frequency that the frequency may change with time. In some cases, the one or more cycles of the waves may be a composite waveform including multiple frequency components. It should be noted that the waveforms as shown in FIG. 1 are for illustration purpose only.

The waveform may be in any suitable waveforms to achieve a uniform membrane stress on the cell. In some cases, the waveform may approximate a sine wave. When such force is applied to the cells as shown in the example 110, the cell shape changes from a lateral ellipsoid shape to a longitudinal shape and then springs back in a cyclic manner while the stress on the membrane may remain relatively constant. In some cases, the waveforms may be determined based on the type of target cells. For instance, the waveforms delivered to the target tissue may be different according to the different types of cells (e.g., breast cancer cells, fibrosarcoma cells, ovarian carcinoma cells, etc.). In some cases, the waveforms delivered to the target tissue may be dependent on the mechanical properties of the target cell (e.g., dynamic and kinematics properties, inertia of a cell, resonant frequency, etc.).

The waveform applied to the target cells may not be direction-sensitive. The waveform in an optimal frequency range with pre-determined amplitudes (103) may expose target cells to cyclic deformations due to acceleration without exerting constant pressure. For example, when the force is applied to the cells at a relatively low frequency (e.g., 20-250 kHz), the cell shape changes from a lateral ellipsoid shape to a longitudinal shape then springs back in a cyclic manner while the stress on the membrane may remain relatively constant. Such cyclic forces may also be referred to as diffuse forces as they do not have specific direction. This may beneficially allow for a treatment operated with high tolerance/low sensitivity to the wave direction.

In optional cases, the waveform may be determined such that the frequency of the force imparted on the target cells may be at around the resonant frequency of the target cell or a sensitive molecule (e.g., heavy protein) in the signaling pathway. In such cases, the amplitude of the deformation of the cell or the sensitive molecule (e.g., cyclic or oscillation movement) may be proportional to the amplitude of the force and to the mechanical amplification factor of the mass at the resonant frequency (although not necessarily linearly proportional). For instance, when a cyclic force is applied at a frequency at or near the resonant frequency of the cell such as around 20 kHz, the deformation of the target cell may be amplified. Similarly, when the cyclic force is applied at the frequency at or near the resonant frequency (e.g., about 15-40 MHz) of the heavy protein that can be mechanically activated and respond to mechanical forces, the deformation of the protein may be amplified.

In some cases, substances may be utilized to lower the resonant frequency of the entire cell or the sensitive molecules (e.g., by increasing inertia of cells and proteins) such that a lower frequency for the cyclic force may be selected. In some cases, the wave may be a single frequency sinusoidal wave that is at or near the resonant frequency of the cell. In some cases, the wave may comprise multiple frequency components that one of them may be at or near the resonant frequency of the sensitive molecule. In some cases, at least one of the multiple frequency components may be higher than the frequency of the cyclic force imparted on the target cells/internal tissues. For instance, the waves may be generated by bursts of high frequency ultrasound such that the periodic forces imparted on the target cells are at the frequency of the bursts (i.e., frequency component with greater amplitude) which can be lower than the frequency of the high frequency component within the burst.

In some cases, the waveform (100) experienced at the site of the target tissue or target cell may have frequency in an optimal range based on the mechanical properties of the target cell as described above. For example, the frequency may be in the range from about 5 kHz to 30 kHz, 30 kHz to about 100 kHz, 30 kHz to about 150 kHz, 30 kHz to about 200 kHz, 30 kHz to about 250 kHz, 40 kHz to about 100 kHz, 40 kHz to about 150 kHz, 40 kHz to about 200 kHz, 40 kHz to about 250 kHz, 50 kHz to about 100 kHz, 50 kHz to about 150 kHz, 50 kHz to about 200 kHz, 50 kHz to about 250 kHz, 60 kHz to about 100 kHz, 60 kHz to about 150 kHz, 60 kHz to about 200 kHz, 60 kHz to about 250 kHz, 70 kHz to about 100 kHz, 70 kHz to about 150 kHz, 70 kHz to about 200 kHz, 70 kHz to about 250 kHz, 30 kHz to about 210 kHz, 30 kHz to about 220 kHz, 30 kHz to about 230 kHz, 30 kHz to about 240 kHz, 40 kHz to about 210 kHz, 40 kHz to about 220 kHz, 40 kHz to about 230 kHz, 40 kHz to about 240 kHz, 50 kHz to about 210 kHz, 50 kHz to about 220 kHz, 50 kHz to about 230 kHz or 50 kHz to about 240 kHz, 150 kHz to 1 MHz, and levels of ultrasound frequency within these stated amounts.

In some cases, the waveform transmitted to and experienced at the site of the target tissue or target cells may be in a low intensity range. For example, low intensity may be no more than 450 mW/cm², 400 mW/cm², 350 mW/cm², 300 mW/cm², 250 mW/cm², 200 mW/cm², 150 mW/cm², 100 mW/cm², 50 mW/cm², mW/cm², 10 mW/cm², or any number below 450 mW/cm² or above 10 mW/cm².

In some embodiments of the present disclosure, the low intensity force may be delivered by modulating the duty cycle and/or amplitude (103) of the force. In some cases, the amplitude (103) of the force may be sufficient to induce cell shape deformations by certain amount without introducing mechanical damage to the target cells or normal cells. For example, the target cell or normal cell shape may be deformed by from about 1% to about 5%, from about 3% to about 8%, from about 5% to about 10% under such cyclic force. Given different sizes of the cells in the target tissue, the deformation may range from 0.1 micron to 12 microns. The duty cycle may be selected such that the deformation is cyclic rather than static. For instance, within each cycle, the on-off ratio of force pulses may be about 5%, 10%, 15%, 20%, 25%, 30%, 35%, 40%, 45%, 50% and the like. Duty factor may be defined as a percent of time that the signal/force is “on” (e.g., transmitted) within a cycle. For example, as illustrated in FIG. 1, the duty factor may refer to the ratio between the pulse duration T1 and the time of a cycle T2. The intensity or the amplitude of the periodic waveform/force may be determined such that no thermal damage is introduced in the target tissue or to the subject.

The cyclic force or waveform may be applied to the target tissue or cells over a period of time (101). The period of time may be in the range of hours. For example, in particular treatment sessions, waveforms may be delivered to the target tissue for about 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 15 hours, 20 hours, 24 hours, 48 hours or more. A treatment session may refer to exposure to the radiation continuously or intermittently. A treatment session may include one or more sub-sessions that may or may not utilize the same cyclic force or waveform. In some cases, the treatment may be repeated for the same or a different length of time, one or more times for days, weeks, months, years, or for the life of the subject.

In some cases, the one or more cycles of waves may be applied to the target tissue/cell directly. In some cases, the one or more cycles of waves are applied to the target cell through a tissue comprising normal cells. In the case when the one or more cycles of waves are applied to the target cell through a tissue, the one or more cycles of waves may not induce spontaneous cell death of the normal cells in the tissue. In some cases, the target cell may be in the body of a subject such as in an internal organ or tissue. In the case when the one or more cycles of waves are applied to the target cell through the body of the subject, the one or more cycles of waves may not induce spontaneous cell death of the normal cells in the subject surrounding the target cells or co-localized with the target cells.

The wave produced as a direct output of a waveform generator or energy source can be in any suitable forms. The wave can be produced by any suitable energy sources so as to induce desired stress or force in the target cells. In some embodiments, the wave may be a mechanical wave that can induce stress or force in the target cells. Mechanical waves that can be used in the present disclosure include but are not limited to acoustic waves. In some embodiments, the mechanical wave may be an ultrasound wave. In some embodiments, the mechanical wave may be a focused ultrasound wave. In some embodiments, the wave used in the present disclosure may be an electromagnetic wave. The electromagnetic wave may include but is not limited to radio waves, microwaves, infrared, (visible) light, ultraviolet, X-rays, gamma rays and the like. In some embodiments, the wave may be a shockwave. The wave produced may be in the form of focused ultrasound, unfocused ultrasound, sweeping ultrasound, shock waves, far-infrared light, microwave or radio frequency electromagnetic waves or any combination of the above. The waves may be pneumatic or hydraulic pressure applied to the target organism.

The wave may be generated by devices or energy sources such as an ultrasound generator, ultrasound transducer, microwave amplification by stimulated emission of radiation (MASER) generator, magnetic resonance imaging (MRI) device, positron emission tomography (PET) device, near infrared light sources, large area generators, neutral generators and various others. Details about the various apparatuses and devices are described later herein.

As used herein, the plurality of characteristics of the waves or cyclic force such as intensities, amplitude and frequencies are the intensity, amplitude and frequency levels at the effective tissue site, not the actual output value of the wave generator (e.g., ultrasound transducer). In some cases, one or more characteristics of the wave as direct output from a generator may be different from those of the cyclic force effective at the target tissue or target region. For example, the waveform transmitted to and experienced at the site of the target tissue or target cells may be a cyclic force at a low-frequency (e.g., 20-250 kHz) and low intensity levels (e.g., <500 mW/cm²). The output of the ultrasound transducer for generating such waveform may have a greater intensity level than the resulting effective amount at the target tissue site to account for energy loss during transmission.

For example, a certain amount of energy may be absorbed by the biologic tissue (e.g., skin, bone, muscle, and underlying fascia) while the ultrasound pulses penetrate such biologic tissue until reaching the target tissue/region. Due to the low frequency characteristic of the cyclic force, the energy loss may be low and the penetration depth may be long. As an example, no more than 5%, 10%, 15%, 20%, 25%, 30%, 35%, 40%, 45%, 50%, 55%, 60%, 65% of energy may be absorbed when transmitting the waves, and the penetration may reach a depth of at least about 0.1, 0.5, 1, 2, 3, 4, 5, 6, 7, 9, 10 cm or more into a human body. Moreover, a benefit from the low frequency and low intensity characteristics of the cyclic force may be the low heat generated as a result of the wave penetrating tissues.

In some cases, the frequency of the wave output from a wave generator may be the same as that of the effective wave at the target cells. For example, the mechanical wave directly outputted from a wave generator may have a frequency in a range of from about 20 to 250 kHz.

The one or more characteristics of the output wave from a specific source may be determined based on factors such as the characteristics of the desired force effective at the target site, the energy source type, device set up, diagnostic information (e.g., type of cancer, location of target region, penetration depth, volume of target region), tissue and organ properties related to force transduction (e.g., acoustic impedance), and various other factors. In some cases, the one or more characteristics of the output wave generated by the wave generator (e.g., ultrasound transducer) may be determined automatically based on the location of the target tissue, the type of target cells, and/or a simulation result. The parameters (e.g., frequency, amplitude, duty-cycle, etc.) for producing the ultrasound pulses may be determined based on empirical data or mechanical simulation of how target cells deform in response to certain frequencies, amplitude at a target location. Alternatively, as the treatment has high tolerance/low sensitivity to the wave direction, and target location (due to the minimal side effects and low energy transmission loss), the waves can be safely applied to a subject without a precise location of the target tissue.

The terms “target zone”, “target region”, “target treatment region”, “treatment region” and the like are used interchangeably throughout the specification unless context suggests otherwise. In some cases, the target region may be represented by a precision location and/or volume. In some cases, the target region may be a rough region that encompasses a diseased tissue or target cell without precise boundary. The provided treatment or therapy may be effective without knowing the precision location/volume of the target region or the target tissue.

In some cases, the one or more cycles of the waves applied to a target region may have constant frequency. In some cases, the one or more cycles of the waves may have variable frequency that the frequency may change along with time. The frequency of the wave may be adjusted dynamically in response to real-time feedback information. Alternatively or in addition to, the frequency of the wave may be adjusted according to a pre-determined treatment plan. In some cases, the one or more cycles of the waves may be a composite waveform including multiple frequency components.

The frequency of the waves may be determined based on the types of the cells. In some cases, the frequency of the wave may be determined based on the volume of the treatment region. In some cases, the frequency of the wave may be determined based on the tissue (e.g., physical properties of the tissue) where the target cells reside. In some cases, the frequency of the wave may be determined based on the subject (e.g., age, gender, personal preference) with the target cells. In some cases, the frequency of the wave may be determined based on the diagnostic information of the subject. The diagnostic information may include, for example, the organ being affected, size of the loci, location of the tissue to be treated, stage of the disease, the state of metastasis and various other information. The diagnostic information may be obtained with aid of any suitable imaging modalities. For example, the internal tissues may be imaged for generating a treatment plan, using a remote imaging modality such as computed tomography (CT), magnetic resonance imaging (MRI), ultrasound imaging, X-ray imaging, optical coherence tomography, bone scan, positron emission tomography (PET) scan, a combination of these, or other imaging modalities. The diagnostic information may not include information about the precise location of the diseased tissue or volume of the diseased tissue. The provided treatment may be effective without knowing the precision location/volume of the target tissue. The diagnostic information can be obtained with aid of any other diagnostic means such as biopsy.

In some embodiments, the one or more cycles of the waves may be delivered with constant intensity. Alternatively or in addition to, the one or more cycles of the waves may be delivered with variable intensities that may change along with time. The intensity of the wave may be adjusted dynamically in response to real-time feedback information. Alternatively or in addition to, the intensity of the wave may be adjusted according to a pre-determined treatment plan.

The intensity of the wave may be determined based on the types of the cells. In some cases, the intensity of the wave may be determined based on the volume of the treatment region. In some cases, the intensity of the wave may be determined based on the tissue (e.g., physical properties of the tissue such as acoustic impedance) where the target cells reside. In some cases, the intensity of the wave may be determined based on the subject (e.g., age, gender, personal preference) with the target cells. In some cases, the intensity of the wave may be determined based on the diagnostic information of the subject. The diagnostic information may include, but is not limited to, the organ being affected, size of the loci, location of the tissue to be treated, stage of the disease and the state of metastasis. The diagnostic information can be obtained with aid of any suitable imaging modalities. For example, the internal tissues may be imaged for generating a treatment plan, using a remote imaging modality such as a computed tomography (CT), magnetic resonance imaging (MRI), ultrasound imaging, X-ray imaging, optical coherence tomography, bone scan, positron emission tomography (PET) scan, a combination of these, or other imaging modalities. The diagnostic information can be obtained with the aid of any other diagnostic means such as biopsy.

The method of the present disclosure can be performed at any appropriate temperature. In some embodiments, the method can be performed at temperature between 4° C. to 45° C. In some embodiments, the method can be performed at temperature between 16° C. to 20° C. In some embodiments, the method can be performed between 20° C. to 40° C. In some embodiments, the method can be performed between 36 to 40° C. For example, the method can be performed at 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45° C.

The one or more characteristics as described above may be determined and specified in a treatment plan. For instance, a treatment plan may comprise information about the cyclic force (e.g., frequency, intensity, amplitude, duty factor, etc.) to be delivered to a target region, information about the treatment region (e.g., location, volume, tissue type, etc.), operation settings (e.g., temperature control), treatment duration (e.g., treatment session duration), or other conditions.

A treatment plan may be generated in a fully automated, semi-automated, or manual manner. In some cases, the treatment plan may be generated automatically upon receiving a diagnostic input. One or more of the characteristics may be determined automatically based on the aforementioned factors. In some cases, the treatment plan may be generated using AI techniques such as machine learning methods. For instance, machine learning models may be trained for generating a treatment plan. In some cases, the input data supplied to the machine learning model may include diagnostic information, device information, personal information or others as described elsewhere herein. In some cases, the output of the machine learning model can be a treatment plan or one or more parameters of the treatment (e.g., characteristic of forces, device setup, treatment duration, etc.). The treatment plan may dynamically adapt to real-time conditions based on feedback information. Alternatively or in addition to, the treatment plan may run through the entire course without real-time feedback information.

In some cases, the machine learning methods used for generating the treatment plan may comprise one or more machine learning algorithms. Examples of machine learning algorithms may include a support vector machine (SVM), a naïve Bayes classification, a random forest, a deep learning model such as neural network, feedforward neural network, radial basis function network, recurrent neural network, convolutional neural network, deep residual learning network, or other supervised learning algorithm or unsupervised learning algorithm.

In some embodiments, the one or more programmed cycles of waves may be transduced to the subject through a medium. The medium can be any medium that can transduce the wave of the present disclosure. In some embodiments, the medium may be a liquid. In some embodiments, the medium may be a gel. In some cases, the medium may be used beneficially for ultrasound coupling to intended tissues to be treated. This may be in addition to or instead of being used for cooling. In some cases, instead of ultrasound coupling gel, other coupling means such as a gel pad may be utilized to couple a transducer to a surface to be treated.

In some cases, booster elements may be utilized to facilitate or enhance cell response to the cyclic force. The booster elements may include using substances such as chemicals or particles that can amplify the local availability or conversion of sound or electromagnetic radiation. The substances may be administered to the target tissue (e.g., injected to tumor) and applied to the external surface interfacing the transducer. The substances may be contained in any suitable elements known in the art such as patches, bubbles, tubes, or balloons.

In some cases, the booster element may include a chemical that may enhance the efficiency of cell signaling. For example, the chemical may lower the resonance frequency of the entire cell or the sensitive molecules (e.g., by increasing inertia of cells and proteins) such that the cell may have a greater response to lower frequency cyclic forces. In some cases, the chemical may affect cell signaling. The chemical can be calcium channel activators to increase calcium influx-induced death thereby enhancing the efficacy of the therapy using cyclic force. For instance, such chemicals can be administered to the target tissue in conjunction with applying the cyclic force to the target region.

### Devices and Systems

In another aspect, the present disclosure provides devices or systems for inducing spontaneous cell death of a target cell by remote cyclic forces. As described above, various types of wave generators or energy sources can be used to generate the aforementioned structured wave. For instance, the structured wave may be generated by devices or energy sources such as ultrasound generator/transducer, microwave amplification by stimulated emission of radiation (MASER) generator, magnetic resonance imaging (MRI) device, positron emission tomography (PET) device, near infrared light sources, large area generators, a customized wave generator, and various others.

The devices may comprise energy sources or wave generators configured to produce one or more programmed cycles of waves to the target cell for a pre-determined period of time. The programmed cycles of waves can be the same as the cyclic waves describe elsewhere herein. For instance, the programmed cycles of waves may be at a frequency in a range of 5-30 kHz, 30-250 kHz, or 150 kHz to 1 MHz with low intensity and high amplitude.

The devices or systems may be configured to produce waves that can induce stress or force in the target cells. The waves can be mechanical waves (e.g., acoustic waves, ultrasound waves). In some cases, the mechanical wave may be a focused ultrasound wave. In some cases, the wave may be an electromagnetic wave. The electromagnetic wave may include but is not limited to radio waves, microwaves, infrared, (visible) light, ultraviolet, X-rays, and gamma rays. In some embodiments, the wave may be a shockwave. The wave produced may be in the form of focused ultrasound, unfocused ultrasound, sweeping ultrasound, shock waves, far-infrared light, microwave or radio frequency electromagnetic waves and others.

In some embodiments, the device may be an ultrasound device comprising one or more ultrasound transducers. The one or more ultrasound transducers may produce one or more programmed cycles of waves to the target cell for a pre-determined period of time. The programmed cycles of waves can be the same as describe elsewhere herein. For instance, the programmed cycles of waves may be at a frequency in a range of, for example, 5-30 kHz, 30-250 kHz, or 150 kHz to 1 MHz with low intensity and high amplitude.

Below lists a few examples of devices designed for delivering the aforementioned structured waves:

### Example 1 Single Element or Area Ultrasound Transducer

In some cases, an ultrasound device may include a single element or area ultrasound transducer. In some cases, an array of ultrasound transducers may be utilized. The array of ultrasound transducers may operate concurrently to generate a sequence of force pulses. The number of transducers may be any number such as a number from 1 to 1000, and may have different amplitudes and/or a phase relationship. For instance, adjacent transducers may have a constant progressive phase shift or variable phase shift thereby adjusting the beam/wave direction. In some cases, the frequencies of the transducers used may be in a simple design, such that all frequency ranges are the same, or may be in a complex design, in which different transducers have different frequency ranges thereby providing a composite waveform including multiple frequency components. The ultrasound may be focused or unfocussed. In some cases, when focused ultrasound is preferred, additional elements such as acoustic lenses or reflecting mirrors may be utilized. The focal plane or focal length of the transducer (array) may be adjusted to direct the beam to the location of a target region. Alternatively, the ultrasound may not be focused. Given that the response of the cancer cells and normal cells to the cyclic force is not sensitive to the direction or orientation of the diffusion force, an unfocussed ultrasound wave can induce desired cyclic stress on the target cells so long as the penetration depth is long enough. One or more characteristics of the wave such as frequency, duty factor, amplitude and the like may be modulated by controlling the one or more ultrasound transducers.

### Example 2 Complex Ultrasound Generators

In some examples, the ultrasound device may comprise an array of individually controlled transducers that allows for beam steering and focusing. Beamforming techniques such as phased array beamforming or beam control methods such as using mirrors of moving acoustic prisms or lenses for adjusting focal length of the device may be utilized. The array of transducers may operate collectively to generate a waveform and transmit the waveform to a target location/region. In addition to the focal length of the ultrasound device, one or more characteristics of the wave such as frequency, duty factor, amplitude and the like may be modulated by controlling the array of ultrasound transducers.

### Example 3 Microwave Amplification by Stimulated Emission of Radiation (MASER) Generator

In some embodiments, the device or system may include a MASER generator. The direct output of the MASER generator may be high intensity and high frequency irradiation which is transmitted into the tissue in a pulsatile or modulated fashion and is partially absorbed by the tissue. The resultant heat shock during the absorption, may lead to a mechanical signal which can be converted to mechanical forces applied to the target cells.

### Example 4 Near Infrared Light Sources

Similar to example 3, in some cases, devices and systems of the present disclosure may transmit pulsed light generated from near infrared laser or high power incoherent light source to tissues. At least a portion of the energy may be absorbed in the tissue and create mechanical stress to the target cells. For instance, gas-discharge lamps such as xenon discharge tubes may be used to create desired structured stress in the biological tissues.

### Example 5 Shockwave Transducer

In some cases, devices and systems of the present disclosure may utilize shockwave to induce structured force to target cells. For instance, broad band pulses from spark discharge, capacitive discharge, and high power transducers, may be delivered to a subject and transmit forces with phonons. By generating the shockwave in a cyclic manner, a cyclic force can be transmitted and applied onto the target cells.

### Example 6 Resonant Stress Generator

In some cases, the provided devices and systems may be configured to generate ultrasound waves at the resonant frequency of a molecule such as a heavy protein that can be mechanically activated and respond to mechanical forces. The resonant frequency may be in the range of 1 MHz to 50 MHz. Such a frequency may open signaling channels and single molecules by disrupting the diffusion, transport, or the transcription of the molecules.

### Example 7 Large Area Generator

In some cases, the provided devices or systems may utilize a large area generator. The large area generator may be preferred in the situations when a large portion of subject is to be treated and lower irradiation is desired. For example, when at least 5%, 10%, 15%, 20%, 25%, 30%, 35%, 40%, 45%, 50%, 55%, 60%, 65% of a subject to be treated, large area generator may be utilized to deliver waves to such fraction of the subject simultaneously. In the case when a large area generator is utilized, the power flux may be relatively low and the exposure time may be longer. The large area generator may include a single large area transducer or antenna arrays that allow for irradiating a substantial fraction of a subject.

### Example 8 Neutral Generator

In some cases, a neutral generator may be utilized to provide additional user safety to the devices and systems. For example, with use of transducer or antenna arrays whose feed net voltages add up to zero or near zero, safe human contact may be provided even in the case of a failure of the insulation. The array of antenna elements can be built with alternating coil direction or transducers with inverted piezo crystals thereby allowing for a zero net voltage. Such alternating direction or inverted piezo crystals design can be applied to one or more pairs of the antenna elements or the entire antenna array.

In some embodiments, the devices and systems may further comprise an element or feature to boost the transduction of the one or more programmed cycles of waves. The booster element for the purpose of the present disclosure may be to increase the transduction of the wave locally without raising the overall intensity. The booster elements may include using substances such as chemicals or particles that can amplify the local availability or conversion of sound or electromagnetic radiation. The substances may be administered to the target tissue (e.g., injected to tumor) and applied to the surface interfacing the transducer. The substances may be contained in any suitable elements known in the art such as patches, bubbles, tubes, or balloons. In some embodiments, the element to boost the transduction of the sequence of programmed cycles of waves may be optical elements or structures for controlling the waves to be transmitted to the target tissue. For example, a wave mirror allowing to direct and/or focus waves and to generate standing wave patterns may be utilized. Any suitable elements such as reflectors, lenses and other optical elements can be used to collimate, concentrate or otherwise modify the beam or irradiation properties of the ultrasound.

### Treatment

In some embodiments, the present disclosure provides a method for treating cancer in a subject in situ. The treatment may trigger the apoptosis induced-cancer cell death in an automated and controlled manner. The provided in situ cancer treatment can be well suited for various kinds of cancer. The treatment involves generating and imparting periodic forces to diseased tissue to introduce apoptosis only in cancer cells while normal cells are not damaged. The treatment may comprise applying sequence of programmed cycles of waves of the present disclosure to the subject for a pre-determined period of time (e.g., hours). The frequency of the wave may be in a range of, for example, 5-30 kHz, 30-250 kHz, or 150 kHz to 1 MHz kHz with low intensity and high amplitude.

In some embodiments, the subject may be diagnosed with cancer. In particular, the cancer includes but is not limited to prostate cancer, breast cancer, colon cancer, lung cancer, head & neck cancer, brain cancer, bladder cancer, lymphoma, ovarian cancer, renal & testis cancer, melanoma, liver cancer, cervical cancer, pancreatic cancer or gastrointestinal cancer.

In some embodiments, the subject is applied with the treatment method of the present disclosure before a surgery. In some embodiments, the subject is applied with the treatment method of the present disclosure after a surgery.

In some embodiments, the treatment method of the present disclosure can be used in conjunction with other treatments or therapies. Examples of the additional treatments or therapies may include, but are not limited to, chemotherapy, radiotherapy, immunotherapy and the like.

The treatment may, in some cases, comprise generating a treatment plan based at least in part on diagnostic information, and executing the treatment plan with aid of the devices and systems as described above. As described elsewhere herein, a treatment plan may comprise information about the cyclic force (e.g., frequency, intensity, amplitude, duty cycle, etc.) to be delivered to a target region, information about the treatment region (e.g., location, volume, etc.), operation settings (e.g., temperature control), treatment duration, or others.

The treatment plan can be generated in a fully automated, semi-automated, or manual fashion as described above. In some cases, the treatment plan may be generated automatically upon receiving a diagnostic input. One or more of the characteristics may be determined automatically based on one or more factors as described above. In some cases, the treatment plan may be generated using AI techniques such as machine learning methods. For instance, machine learning models may be trained for generating a treatment plan. In some cases, the input data supplied to the machine learning model may include diagnostic information, device information, or personal information. In some cases, the output of the machine learning model can be a treatment plan or one or more parameters of the treatment plan (e.g., characteristic of forces, device setup, treatment duration, etc.). The treatment plan may dynamically adapt to real-time conditions based on feedback information. Alternatively or in addition to, the treatment plan may run through the entire course without real-time feedback information.

In some cases, the machine learning method used for generating the treatment plan may comprise one or more machine learning algorithms. Examples of machine learning algorithms may include a support vector machine (SVM), a naïve Bayes classification, a random forest, a deep learning model such as neural network, feedforward neural network, radial basis function network, recurrent neural network, convolutional neural network, deep residual learning network, or other supervised learning algorithm or unsupervised learning algorithm.

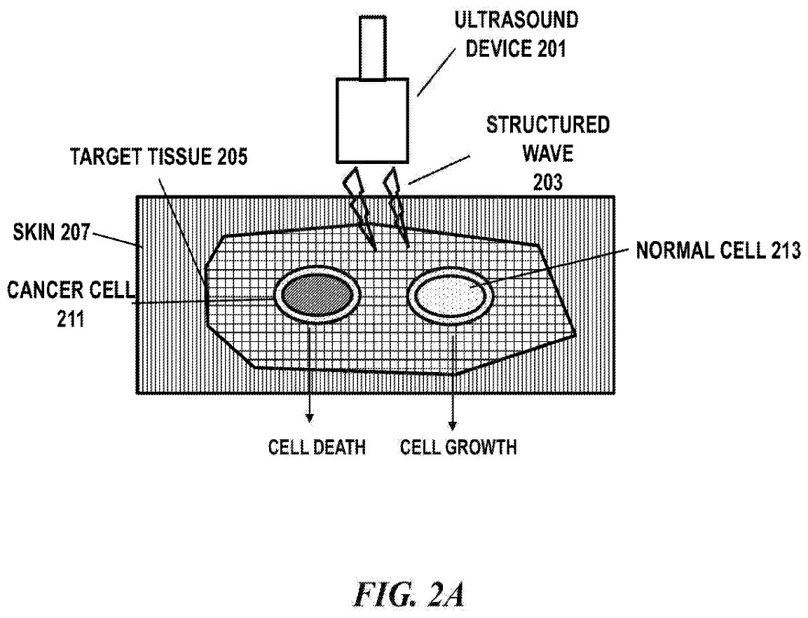

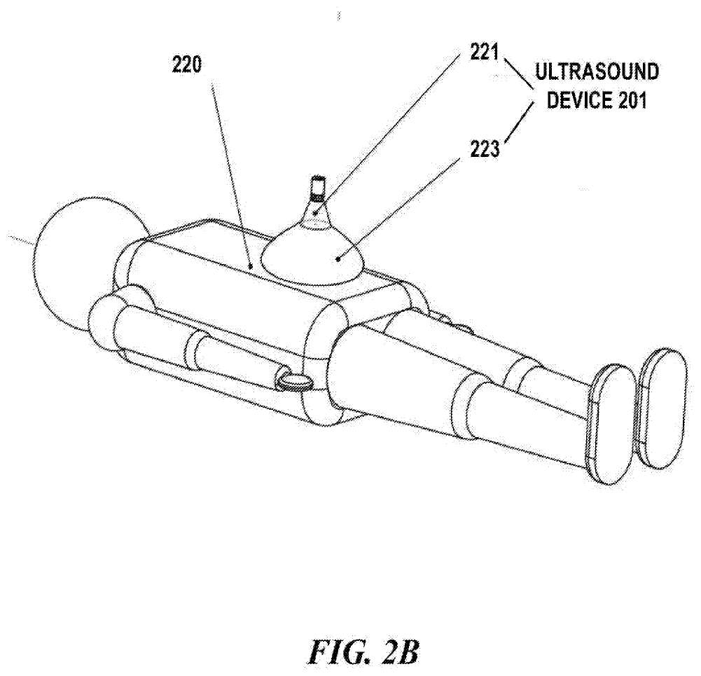

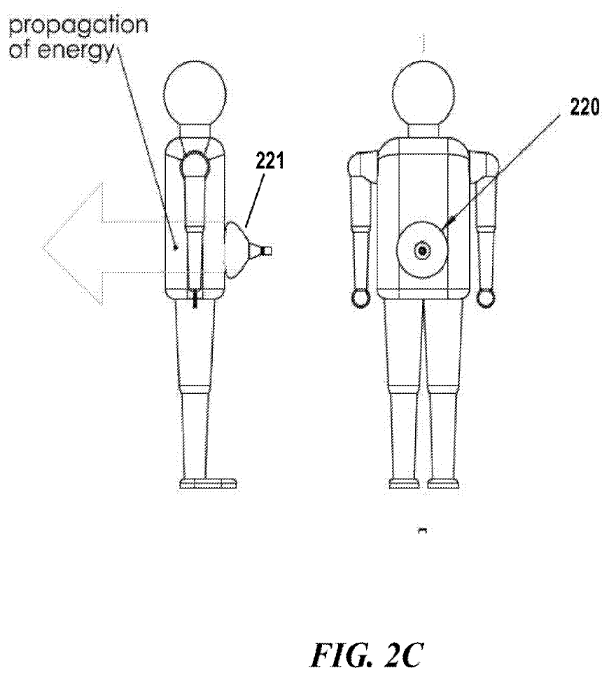

FIGS. 2A-2C shows an example of a device (201) for treating cancer implementing a method provided herein. The device may be an ultrasound device (201). The ultrasound sound device may comprise one or more ultrasound transducers (221). The one or more ultrasound transducers may be configured to generate cycles of waves (203) to be delivered to a target tissue (205). In some cases, the target tissue (205) may be an internal tissue that may require the waves penetrate through at least a portion of the subject (e.g., skin 207).

The ultrasound device (201) may comprise one or more transducers (221) configured to generate structured waves (203) as described elsewhere herein. The structured waves may be ultrasound waves that can be of any shape, and can be focused or unfocused. The ultrasound transducer(s) may be single element or area ultrasound transducer, complex ultrasound generator or any other types as described elsewhere herein. In some cases, the one or more transducers may be a neutral generator to provide additional user safety.

The structured waves (203) may penetrate through biological tissues such as skin (207), bone, muscle, or underlying fascia to reach the target tissue (205). The structured waves (203) may be mechanical waves having a low frequency such as in the range of 5-30 kHz, 30-250 kHz, or 150 kHz to 1 MHz kHz. The structured waves (203) may be capable of penetrating into the tissue at a depth of at least about 0.1, 0.5, 1, 2, 3, 4, 5, 6, 7, 9, 10 cm or more to induce sufficient cyclic stress to the cells in the target tissue (205). The force transmitted to and exerted on the cells may be a cyclic force. The cyclic force can be the same as the cyclic force as described with respect to FIG. 1. For example, the cyclic force may be at a low-frequency (e.g., 20 to 250 kHz) and low intensity levels (e.g., <500 mW/cm²). The cyclic force applied to the cells in the target tissue (205) may have high amplitude to induce sufficient cyclic deformation of the cells in the target tissue.

The target tissue (205) may be tumor tissue, the cell death pathway of cancer cells (211) may be activated and spontaneous cell death may be further induced upon administration of the structured force (203). Meanwhile, normal cells (213) may not suffer from damage or negative side effect. In some cases, cell growth may be promoted in the normal cells as part of regeneration/healing (213).

In some embodiments, the one or more programmed cycles of waves may be transduced to a treatment contact area of a subject through a medium. The medium can be any medium that can transduce the wave of the present disclosure. In some embodiments, the medium may be a liquid. In some embodiments, the medium may be a gel. In some cases, the medium may be used beneficially for ultrasound coupling to intended tissues to be treated. This may be in addition to or instead of being used for cooling. In some cases, instead of an ultrasound coupling gel, other coupling means such as a gel pad may be utilized to couple a transducer to a surface to be treated.

As illustrated in FIG. 2B, the ultrasound transducer (221) may comprise a case that is heat conductive and electrically insulated. In some cases, the ultrasound device (201) may comprise an element such as a gel-filled bag displaced at the interface of the case and a treatment surface (220) of the patient for coupling energy to the treatment surface therein thereby facilitating transduction of the ultrasonic waves. The gel-filled bag may be safe to human contact and have an acoustic impedance helpful for coupling ultrasound to the patient. For example, the acoustic impedance may be similar to, or higher than, that of the tissues/skin of the patient to be treated.

In some cases, the ultrasound device can be placed at a treatment area of the subject to be in direct contact with a body surface. As described above, as the treatment has high tolerance/low sensitivity to the wave direction and target location (due to the minimal side effect and low energy transmission loss), the waves can be safely applied to a subject without a precise location of the target tissue so long as the propagation of the energy waves passes through the target tissue, internal organ/tissue as illustrated in FIG. 2C.

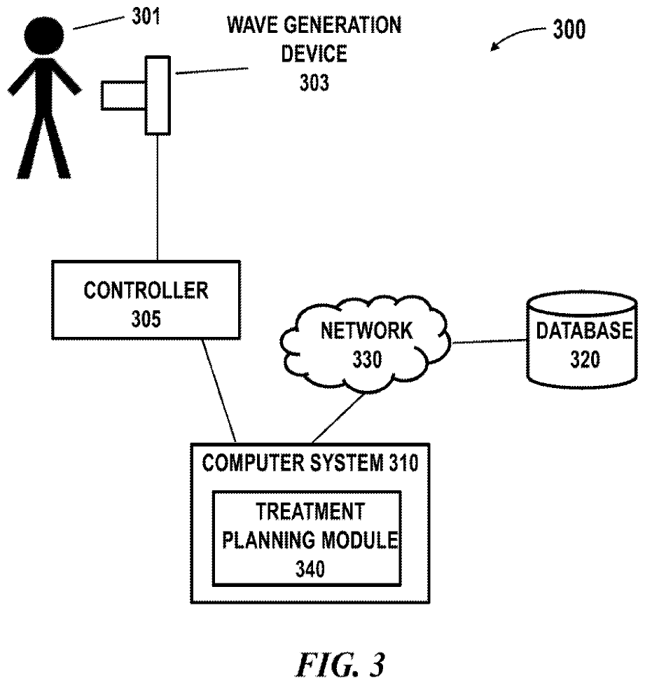

FIG. 3 schematically shows a system (300) in which cancer treatment method described herein can be implemented. The system (300) may comprise a wave generation device (303) for delivering cyclic waves to a subject (301) (e.g., patient) and a controller (305) operably coupled to the wave generation device. In one example, mechanical waves produced by the wave generation device (303) may be delivered to at least a portion of the patient (301). The controller (305) may control the energy source such as ultrasound transducers of the wave generation device (303). The system (300) may further comprise a computer system (310) and one or more databases operably coupled to the controller (301) over the network (330). The computer system (310) may comprise a treatment planning module (340) implementing methods provided herein for generating treatment plans. For example, the computer system (310) may be used for generating a treatment plan based on diagnostic information, personal information, device setup and the like. Although the illustrated diagram shows the controller and computer system as separate components, the controller and computer system can be integrated into a single component.

The controller (305) may be operated to provide the wave generation device controller information about a pulse sequence and/or to manage the operations of the entire system, according to installed software programs. In some cases, the controller may also serve as an element for instructing a patient to perform tasks, such as, for example, align a part of the body to the device by a voice message produced using an automatic voice synthesis technique. The controller may receive commands from an operator which indicate the treatment to be performed. The controller may comprise various components such as a pulse generator module which is configured to operate the system components to carry out the desired wave or cyclic force sequence, producing data that indicate the timing, strength and shape of the wave or ultrasound pulses to be produced, and the direction of the beam. In some cases, the controller (305) may control pulse generator module and/or a set of gradient amplifiers of the wave generation device (303) to control the frequency, amplitude and shape of the pulses or waves to be produced during the treatment. In some situations, the pulse generator module may also receive real-time patient data from a physiological acquisition controller that receives signals from sensors attached to the patient, such as ECG (electrocardiogram) signals from electrodes or respiratory signals from a bellows. The controller (305) may be coupled to various sensors for monitoring the condition of the patient and the wave generation device. For instance, temperature sensor may be coupled to the controller (305) for temperature control during the operation. In some cases, the system (300) may include a patient positioning system that may receive commands to move the patient to the desired location for the treatment. In some cases, the patient positioning system may utilize proximity sensors to detect the location of a body of the patient. The proximity sensors may be the ultrasound device (303) where the ultrasound transducer may be paired with one or more receivers to measure a distance based on time of flight. Alternatively or in addition to, the proximity sensors may be additional sensors such as an additional ultrasonic device or imaging sensor.

In some cases, the controller (305) may comprise or be coupled to an operator console (not shown) which can include input devices (e.g., keyboard) and control panel and a display. For example, the controller may have input/output (I/O) ports connected to an I/O device such as a display, keyboard and printer. In some cases, the operator console may communicate through the network with the computer system (310) that enables an operator to control the treatment procedure or modify a treatment on a screen of display.

The system (300) may comprise a user interface. The user interface may be configured to receive user input and output information to a user. The user input may be related to control of a treatment procedure or generating/modifying a treatment plan. The user input may be related to the operation of the wave generation device (303) (e.g., parameters for controlling program execution such as terminating a treatment procedure, parameters for controlling the waves to be delivered to the target region such as frequencies, amplitude, etc). The user input may be related to various operations or settings about the generating a treatment plan. The user input may include, for example, a selection of a target location, simulation settings for displaying the target location within a human body, selection of a pre-stored treatment plan, modification of one or more characteristics of the waves, information about the patient and various others. The user interface may rendered on a screen such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

The system (300) may comprise computer systems (310) and database systems (320), which may interact with the controller. The computer system can comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, a plurality of processors for parallel processing, in the form of fine-grained spatial architectures such as a field programmable gate array (FPGA), an application-specific integrated circuit (ASIC), digital signal processor (DSP), and/or one or more RISC processors. The processor can be any suitable integrated circuits, such as computing platforms or microprocessors, logic devices and the like. Although the disclosure is described with reference to a processor, other types of integrated circuits and logic devices are also applicable.

The system (300) may comprise one or more databases. The one or more databases (320) may utilize any suitable database techniques. For instance, structured query language (SQL) or “NoSQL” database may be utilized for storing diagnostic data, such as image data obtained by suitable imaging modalities, training datasets or trained model for generating treatment plan, parameters of a treatment plan, historical treatment plan, etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present invention. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

The network (330) may establish connections among the components in the system (300) and a connection of the system to external systems. The network (330) may comprise any combination of local area and/or wide area networks using both wireless and/or wired communication systems. For example, the network (330) may include the Internet, as well as mobile telephone networks. In one embodiment, the network (330) uses standard communications technologies and/or protocols. Hence, the network (330) may include links using technologies such as Ethernet, 802.11, worldwide interoperability for microwave access (WiMAX), 2G/3G/4G mobile communications protocols, asynchronous transfer mode (ATM), InfiniBand, PCI Express Advanced Switching, etc. Other networking protocols used on the network (230) can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for treating target cells in a target tissue, the method comprising:
determining frequency and magnitude of a periodic force to be applied to the target cells to trigger a mechanically-induced apoptotic process in the target cells; and
generating a sequence of programmed cycles of waves to the target tissue to apply the periodic forces to the target cells for a period of time,
wherein a frequency, magnitude and the period of time of the periodic force are determined based at least in part on a type of the target cells or mechanical properties of the target cells.

2. The method of claim 1, wherein the target cells are cancer cells and wherein the periodic force is imparted on both the cancer cells and normal cells surrounding the cancer cells.

3. The method of claim 2, wherein the periodic force promotes or preserves survival and regeneration of the normal cells.

4. The method of claim 1, wherein the frequency of the periodic force is in a range of 30 kHz to 250 kHz.

5. The method of claim 1, wherein the frequency of the periodic force is in a range of 5 kHz to 50 kHz.

6. The method of claim 1, wherein the frequency of the periodic force is in a range of 150 kHz to 1 MHz.

7. The method of claim 1, wherein the target cells are deformed periodically at the frequency for the period of time such that the mechanically-induced apoptotic process is triggered.

8. The method of claim 1, wherein the sequence of programmed cycles of waves are ultrasound waves and are generated by an ultrasound generator.

9. The method of claim 1, wherein the sequence of programmed cycles of waves are generated by at least one of ultrasound generator, ultrasound transducer, microwave amplification by stimulated emission of radiation (MASER) generator, magnetic resonance imaging (MRI) device, positron emission tomography (PET) device, near infrared light source, large area generator, and neutral generator.

10. The method of claim 1, wherein the sequence of programmed cycles of waves are mechanical waves, electromagnetic waves, periodic pneumatic or hydraulic pressure waves.

11. The method of claim 1, wherein the sequence of programmed cycles of waves are a composite waveform including multiple frequency components.

12. The method of claim 11, wherein at least one of the multiple frequency components has a frequency higher than the frequency of the periodic force.

13. The method of claim 1, wherein the mechanical properties of the target cells in the target tissue comprise a resonant frequency of the target cells or an inertia of the target cells.

14. A system for treating target cells in a target tissue, comprising:
(i) an energy source configured to generate a sequence of programmed cycles of waves to the target tissue to apply a periodic force to the target cells for a period of time; and
(ii) one or more processors programmed to determine the frequency and magnitude of the periodic force applied to the target cells to trigger mechanically-induced apoptotic process in the target cells, wherein the frequency, magnitude and the period of time are determined based at least in part on a type of the target cells or mechanical properties of the target cell.

15. The system of claim 14, wherein the target cells are cancer cells and wherein the periodic force is imparted on both the cancer cells and normal cells surrounding the cancer cells.

16. The system of claim 15, wherein the periodic force promotes or preserves survival and regeneration of the normal cells.

17. The system of claim 14, wherein the frequency of the periodic force is in a range of 30 kHz to 250 kHz.

18. The system of claim 14, wherein the frequency of the periodic force is in a range of 5 kHz to 50 kHz.

19. The system of claim 14, wherein the frequency of the periodic force is in a range of 150 kHz to 1 MHz.

20. The system of claim 14, wherein the target cells are deformed periodically at the frequency for the period of time such that the mechanically-induced apoptotic process is triggered.

21. The system of claim 14, wherein the sequence of programmed cycles of waves are ultrasound waves and the energy source comprises an ultrasound generator.

22. The system of claim 14, wherein the energy source is at least one of ultrasound generator, ultrasound transducer, microwave amplification by stimulated emission of radiation (MASER) generator, magnetic resonance imaging (MRI) device, positron emission tomography (PET) device, near infrared light source, large area generator, and neutral generator.

23. The system of claim 14, wherein the sequence of programmed cycles of waves are mechanical waves, electromagnetic waves, periodic pneumatic or hydraulic pressure waves.

24. The system of claim 14, wherein the sequence of programmed cycles of waves are a composite waveform including multiple frequency components.

25. The system of claim 24, wherein at least one of the multiple frequency components has a frequency higher than the frequency of the periodic force.

26. The system of claim 14, wherein the mechanical properties of the target cells in the target tissue comprise a resonant frequency of the target cells or an inertia of the target cells.
